(19) United States
(12) Patent Application Publication
(10) Pub. No.: US 2008/0015708 A1
(43) Pub. Date: Jan. 17, 2008

(54) Hip stem centralizer cover and method

(75) Inventors: Donald W. Howie, Tennyson (AU); David M. Blakemore, Warsaw, IN (US); Kevin S. Cook, Warsaw, IN (US); Stephen H. Hoag, Warsaw, IN (US); Erin M. Johnson, Columbia City, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/487,290

(22) Filed: Jul. 13, 2006

(51) Int. Cl. A61F 2/36

(52) U.S. Cl. 623/23.22; 623/23.48; 606/95

(57) ABSTRACT

A hip stem centralizer and method are provided for positioning a proximal portion of a femoral hip stem component at a desired radial position within an intramedullary canal of a femur. The hip stem centralizer includes a resilient opening that is elastically compressed upon receiving the hip stem to bias the hip stem to a desired position in the intramedullary canal.

### FIELD OF THE INVENTION

The present invention relates to total hip arthroplasty. More particularly, the present invention relates to a proximal centralizer for a cemented hip stem and a method for its use.

### BACKGROUND

Total hip arthroplasty is often used to restore function to a diseased or injured hip joint. Positions and directions relative to the hip joint may be described in terms of proximal being nearer the hip joint, distal being further from the hip joint, anterior being nearer the front of the body, posterior being nearer the back of the body, medial being nearer the centerline of the body, and lateral being further from the center line of the body. In total hip arthroplasty (FIGS. 1-2), the articular surfaces of the femur and pelvis are cut away and replaced with prosthetic implant components. In a typical case, the implants include a hip stem component 10, a femoral head component 12, an acetabular component 14, and bone cement 16. The hip stem component includes a stem portion 18 extending down into the intramedullary canal 20 of the femur 22 and a neck portion 24 extending away from the femur 22 to support the femoral head component 12.

The femur 22 is prepared by reaming the intramedullary canal 20 down into the bone along an axis 26 from a proximal position near the hip joint at the upper end of the femur 22 toward a distal position nearer the knee joint at the lower end of the femur 22. The pelvis 28 is prepared by reaming the acetabulum 30. Bone cement 16 is introduced into the prepared intramedullary canal 20 and acetabulum 30 and the prosthetic components are seated in the bone cement 16 so that it hardens around and locks the components in place. Positioning the femoral stem component 10 in the correct orientation within the cement 16 is important for proper biomechanical functioning and long term stability. By way of example, it is desirable to have a uniform and strong cement mantle 16 proximally around the anterior 34, lateral 36, and posterior 38 portions of the stem component 10. Also by way of example, femoral components, especially collarless ones, are sometimes placed at the wrong angle in the mediolateral direction. The typical situation is a varus placement in which the angle between the neck 24 and femoral axis 26 is too shallow.

### SUMMARY

The present invention provides a hip stem centralizer and method for positioning a proximal portion of a femoral hip stem component at a desired radial position within an intramedullary canal of a femur. The hip stem centralizer includes a resilient opening that is elastically compressed upon receiving the hip stem to bias the hip stem to a desired position in the intramedullary canal.

In one aspect of the invention, the hip stem centralizer includes a body having a top, a bottom, and defining a resilient opening from the top to the bottom. The hip stem centralizer further includes an element for positioning the resilient opening at a desired position relative to the femur.

In another aspect of the invention, the hip stem centralizer includes a body having a relatively more rigid outer ring and a relatively less rigid inner ring. The inner ring defines a resilient opening extending through the inner ring and being radially elastically compressible upon receiving the hip stem component. The hip stem centralizer further includes at least one member projecting distally from the body in predetermined radial relationship to the resilient opening.

In another aspect of the invention, a method of positioning a proximal portion of a femoral hip stem component within an intramedullary canal of a femur includes: engaging a hip stem centralizer with the opening of the intramedullary canal, the hip stem centralizer defining a resilient opening; inserting a hip stem component through the resilient opening and into bone cement in the intramedullary canal, the resilient opening compressing to bias the hip stem component toward the center of the resilient opening; and disengaging the hip stem centralizer from the hip stem component once the hip stem component has been positioned.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an anterior sectional view of a total hip prosthesis implanted in a hip joint;

FIG. 2 is a sectional view taken along line 2-2 of FIG. 1;

FIG. 3 is an anterior elevation view of an illustrative hip stem centralizer;

FIG. 4 is a top plan view of the illustrative hip stem centralizer of FIG. 3;

FIG. 5 is an anterior elevation view of the illustrative hip stem centralizer of FIG. 3 in use in conjunction with a optional hip stem inserter to position a hip stem component;

FIG. 6 is an anterior elevation view of the illustrative hip stem centralizer of FIG. 3 in use to position a hip stem component; and

FIG. 7 is a lateral elevation view of the hip stem inserter of FIG. 5.

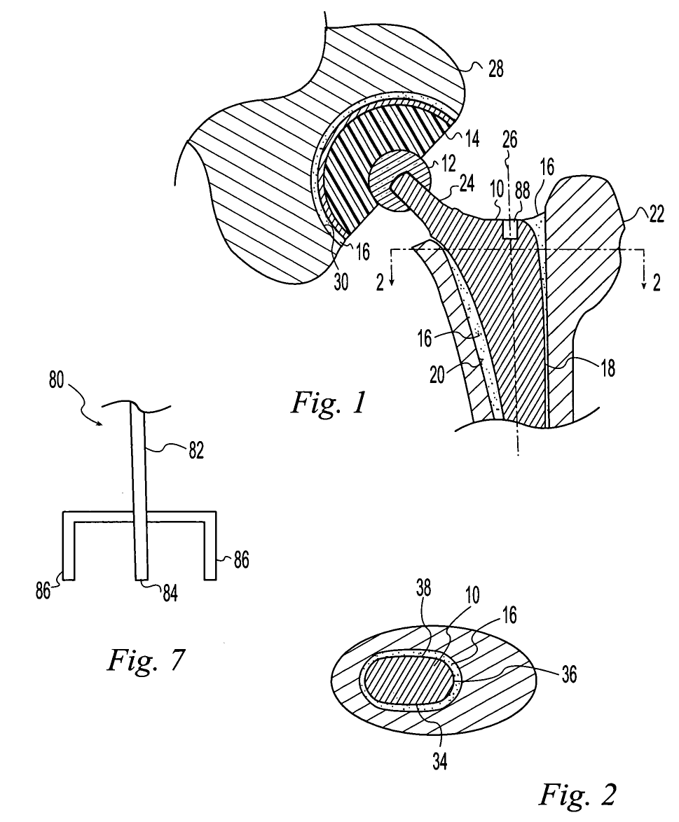

### DESCRIPTION OF THE ILLUSTRATIVE EXAMPLES

Embodiments of a hip stem centralizer include a body defining a resilient opening positionable adjacent to the opening of the intramedullary canal of a femur during hip replacement surgery. A hip stem component may be inserted through the opening and into a mass of bone cement in the intramedullary canal. The sides of the opening may engage the hip stem component and bias it toward a predetermined position in the opening to position the hip stem relative to the canal. If the hip stem component is improperly positioned, it will compress the resilient opening which in turn biases the hip stem component back toward the correct position.

The resilient opening may include springs, films, sheets, foams, and/or other resilient material to bias the opening toward a predetermined position. The resilient opening may be formed in metal, plastic, rubber, and/or other suitable materials. For example the opening may include a metal or polymer spring, polymer film, and/or elastic foam. A foam opening may have a closed cell and/or an open cell configuration. The opening may include a ring of resilient material. For example, the opening may be formed as a ring of elastically compressible closed cell polyethylene foam.

The resiliency of the opening may be tailored by varying the material dimensions and/or physical composition. For example, the material density may be varied to vary the resiliency of the opening. For example a higher density material will yield a more rigid opening while a lower density material will yield a less rigid opening. Likewise, a thicker material may provide more biasing force than a thinner material. The total potential displacement of the hip stem component relative to the opening may be controlled by selecting the thickness of the resilient portion of the opening. For example, a thicker resilient portion will allow for greater total displacement of the hip stem component while a thinner resilient portion will allow for smaller total displacement of the hip stem component. The size of the opening relative to the size of the hip stem component may be selected to control the effective resiliency of the opening and the potential total displacement or the hip stem component. For example, an opening smaller than the hip stem component will be pre-compressed upon insertion of the hip stem component such that it grips the hip stem component and biases it toward the center of the opening with a particular effective resiliency and a particular total potential displacement. By making the opening even smaller relative to the hip stem component, the opening will be pre-compressed more and thus have a more rigid effective resiliency and a smaller total potential displacement.

The body may include a feature for locating the resilient opening relative to the opening of the intramedullary canal. The feature may include projections engageable with the femur. For example the body may include pins, screws, tabs, skirts, and/or other features engageable with the bone. The feature may be engageable with the outer surface of the femur to position the resilient opening relative to the outer surface. The feature may be engageable with holes formed in the femur. For example, holes may be predrilled into the calcar region of the femur to receive pins to position the resilient opening. The feature may be engageable with the inside surface of the intramedullary canal to locate the resilient opening relative to inside surface. For example, the body may include tabs that extend distally to engage the inner edge of the opening of the intramedullary canal to position the resilient opening relative to the inner edge.

The body may include a rigid portion relatively more rigid than the resilient opening to support the resilient opening and aid in positioning the resilient opening. For example the body may include a relatively more rigid outer ring and a relatively less rigid inner ring coupled to the outer ring. The outer ring may support the inner ring to control deformation of the inner ring. The outer ring may positively locate the resilient opening on the bone. The more rigid portion may be made of metal, plastic, and/or other suitable materials. For example, the body may include a rigid plastic outer ring having tabs for locating the ring relative to the intramedullary canal and a resilient foam inner ring for receiving the hip stem component. The resilient opening may be positioned relative to the outer ring to position the resilient opening at any desired position relative to the intramedullary canal. For example the outer ring may engage a lateral portion of the intramedullary canal and the resilient opening may be offset to position the hip stem component at a desired spacing from the lateral portion of the intramedullary canal. The resilient opening may be similarly offset to provide a desired spacing anteriorly, posteriorly, and/or medially.

The hip stem centralizer may seal around the hip stem component and seal the intramedullary canal. Upon insertion of the hip stem component, the seal prevents the cement from exiting the opening of the intramedullary canal such that the bone cement is pressurized into pores in the bone and into close proximity to the hip stem component. The resilient opening may wipe the surface of the hip stem component to reduce air bubbles carried into the bone cement upon insertion of the hip stem component.

The hip stem centralizer may be engageable by a cement injector to allow introduction of bone cement though the hip stem centralizer and into the intramedullary canal of the bone. The hip stem centralizer may seal the intramedullary canal to facilitate pressurization of the bone cement into the intramedullary canal. The hip stem centralizer may also seal the intramedullary canal to prevent cement from exiting the canal and being deposited adjacent the canal opening.

The hip stem centralizer may be engageable with an optional hip stem inserter to orient the hip stem in a desired orientation relative to the hip stem centralizer. For example, the hip stem centralizer may include a first engagement feature and the hip stem inserter may include a complimentary second engagement feature engageable with the first engagement feature. For example, the hip stem centralizer and hip stem inserter may include tabs, grooves, slots, prongs, rods, holes, and/or other suitable keying arrangements such that the hip stem inserter may be engaged with the hip stem in a predetermined relationship and the hip stem inserter may be engaged with the hip stem centralizer in predetermined relationship to orient the hip stem in predetermined relationship to the hip stem centralizer and consequently relative to the femur.

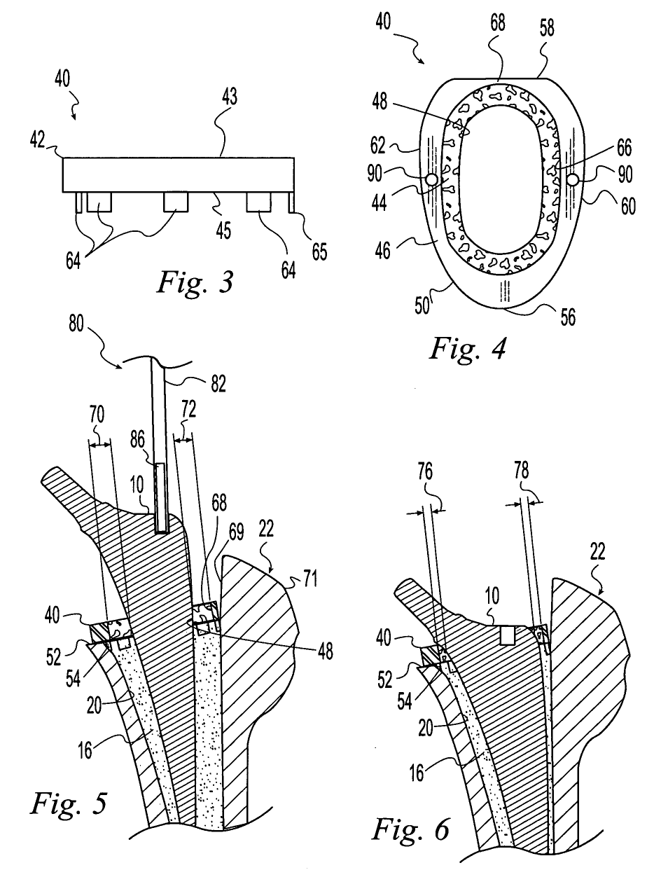

FIGS. 3-6 show an illustrative hip stem centralizer 40 having a body 42 including an inner ring 44 of resilient foam and an outer ring 46 of rigid plastic. The inner and outer rings 44, 46 are bonded together. The inner ring 44 defines a resilient opening 48 through the body 42 from the top 43 to the bottom 45 and sized to receive the hip stem component 10.

The outer ring 46 includes an outer profile 50 shaped to fit on the prepared calcar 52 (FIG. 5) adjacent to the opening 54 of the intramedullary canal 20. The shape of the outer ring 46 can be used to locate the resilient opening 48 relative to the intramedullary canal 20 by aligning the profile 50 with the perimeter of the calcar 52. The outer ring 46 includes a medial side 56, a lateral side 58, an anterior side 60, and a posterior side 62 generally shaped like the medial, lateral, anterior, and posterior sides of the calcar 52. The outer ring 46 further includes tabs 64 extending distally downwardly from the bottom 45 to engage the inner edge of the intramedullary canal opening 54. The resilient opening 48 is spaced radially from the tabs 64 a predetermined distance to space the hip stem component 10 at a desired radial distance from the intramedullary canal 20. For example, a lateral tab 65 extends distally from the lateral side 58 of the outer ring 46 to engage the lateral side of the intramedullary canal 20. The resilient opening 48 is spaced medially from the lateral tab 65 to ensure a minimum bone cement mantle thickness laterally.

As the hip stem component is inserted into the resilient opening 48, it compresses the inner ring 44. The compressed inner ring 44 biases the hip stem toward the center of the resilient opening. The inner ring 44 material may be selected such that when the inner ring 44 is fully compressed, the hip stem component 10 is spaced a desired minimum distance from the intramedullary canal 20. In addition to, or in conjunction with, the fully compressed inner ring 44, the outer ring 46 may have an inner perimeter 66 that overlaps the opening 54 of the intramedullary canal 20 to provide a rigid stop to prevent the hip stem component from approaching nearer than a desired minimum distance from the intramedullary canal 20. In the illustrative hip stem centralizer cap 40, a lateral portion 68 (FIG. 5) of the inner perimeter 66 prevents the hip stem component 10 from being positioned nearer than a desired minimum distance from the lateral portion of the intramedullary canal. In one embodiment, the lateral portion 68 is abutted against the reamed surface 69 of the greater trochanter 71 to position the resilient opening 48 relative to the reamed surface 69.

The outer ring 46 and/or resilient opening 48 may engage a tapering hip stem 10 (as shown) to limit the insertion depth of the stem. For example, as the stem 10 slides through the hip stem centralizer 40, the widening proximal portion of the stem will fit progressively more tightly in the hip stem centralizer 40. At some point, the compressed resilient opening 48 and/or the outer ring 46 may prevent further insertion of the hip stem 10. By appropriately sizing of the openings in the hip stem centralizer 40, the insertion depth of the stem 10 may be controlled.

An inserter 80 (FIG. 7) may be optionally used to aid insertion and orientation of the hip stem 10. The illustrative inserter 80 includes a central shaft 82 having a distal end 84 engageable with a socket 88 in the hip stem component. The socket 88 and distal end 84 preferably have non-circular cross sections so that the hip stem 10 may be coupled to the hip stem inserter in predetermined angular relationship. The inserter 80 facilitates manipulation of the hip stem 10 by providing a tool for pressing the hip stem 10 into the intramedullary canal and moving the hip stem 10 to adjust its position. The inserter 80 further includes a cylindrical outrigger 86 engageable with a bore 90 (FIG. 4) formed in the hip stem centralizer 40 to positively orient the inserter 80, and thus the hip stem 10, relative to the hip stem centralizer 40. In the illustrative embodiment, a pair of outriggers 86 and a pair of complimentary bores 90 are provided.

In use, bone cement 16 is introduced into the intramedullary canal 20. The bone cement 16 may be introduced before or after the hip stem centralizer 40 is positioned on the femur. For example the bone cement may be introduced and then the centralizer 40 may be engaged and aligned with the opening of the intramedullary canal 20 by aligning its outer profile 50 with the calcar 52 and engaging the tabs 64 with the inner edge of the canal 20 opening. This positions the resilient opening 48 at a desired medial/lateral and anterior/posterior position relative to the intramedullary canal 20. Alternatively the lateral portion of the outer ring 46 is abutted with the reamed surface 69 of the greater trochanter 71 to position the resilient opening 48. The hip stem component 10 is inserted through the resilient opening 48 and into the bone cement 16. As the hip stem component is inserted it engages the edges of the resilient opening 48 and begins to compress the inner ring 44. The inner ring 44 is compressed to an initial thickness 70, 72 medially and laterally. The resilient opening also seals the hip stem component 10 and intramedullary canal 20 such that further insertion causes the hip stem component 10 to act as a plunger to pressurize the cement 16 and force it into close engagement with the intramedullary canal 20 and hip stem component 10. Furthermore, as the hip stem component 10 is inserted, the conforming resilient opening wipes the surface of the hip stem component 10 and reduces the amount of air that is entrained as bubbles at the component/cement interface.

Further insertion of the hip stem component 10 compresses the inner ring 44 further causing the inner ring 44 to bias the hip stem component 10 toward the center of the resilient opening 48 and space the hip stem component 10 from the intramedullary canal 20. With the hip stem component 10 fully inserted, the inner ring 44 is compressed to a final compressed thickness 76, 78 (FIG. 6) which strongly biases the hip stem component toward the center of the resilient opening 48. After the bone cement 16 has hardened sufficiently to maintain the hip stem component 10 in the desired position, the hip stem centralizer cap 40 is removed. It may be pulled over the top of the hip stem component 10, cut and pulled radially away, hinged, and/or otherwise removed.

In cases where the optional inserter 80 is employed, the inserter 80 is engaged with the hip stem component 10 and used to press the hip stem component 10 through the hip stem centralizer 40 and into the intramedullary canal 20. As the hip stem component 10 nears its fully seated position, the outriggers 86 of the inserter 80 are engaged with the bores 90 formed in the hip stem centralizer 40 to positively align the hip stem component 10 with the hip stem centralizer 40 and thus with a desired position within the intramedullary canal 20. The length of the outriggers 86 and the depth of the bores 90 are configured to yield a desired insertion depth of the hip stem component 10 when the outriggers 86 are fully seated 90 in the bores 90.

In an alternative method of use, the hip stem centralizer 40 is first positioned over the opening of the intramedullary canal 20 and the resilient opening 48 is engaged with a cement injector. As cement is introduced into the intramedullary canal 20, the hip stem centralizer 40 seals the intramedullary canal 20 and facilitates pressurization of the cement in the canal.

In another alternative method of use, the hip stem centralizer 40 is first engaged with the hip stem component 10 in fully seated relationship and then the hip stem component 10 and hip stem centralizer 40 assembly are introduced to the bone and the hip stem component 10 is inserted until the hip stem centralizer 40 engages the opening of the intramedullary canal 20 and thus establishes the position of the hip stem component 10.

Although examples of a hip stem centralizer cap and its use have been described and illustrated in detail, it is to be understood that the same is intended by way of illustration and example only and is not to be taken by way of limitation. Accordingly, variations in and modifications to the hip stem centralizer cap and its use will be apparent to those of ordinary skill in the art, and the following claims are intended to cover all such modifications and equivalents.

What is claimed is:

1. A hip stem centralizer for positioning a proximal portion of a femoral hip stem component at a desired radial position within an intramedullary canal of a femur, the femoral hip stem component and intramedullary canal each having a proximal portion nearer the hip joint, a distal portion nearer the knee joint, an anterior portion toward the front of the body, a posterior portion toward the rear of the body, and a lateral portion further from the midline of the body, the femur having an outer surface adjacent the proximal portion, the hip stem centralizer comprising:
a body having a top, a bottom, and defining a resilient opening from the top to the bottom, the resilient opening being elastically compressible upon receiving the hip stem component in center biasing relationship toward the center of the opening; and
means for positioning the resilient opening at a desired position relative to the femur, the means for positioning the resilient opening being operably connected to the body.

2. The hip stem centralizer of claim 1 wherein the body comprises a ring of elastically compressible foam defining the resilient opening.

3. The hip stem centralizer of claim 1 wherein the resilient opening is smaller than the proximal portion of the hip stem component.

4. The hip stem centralizer of claim 1 wherein the means for positioning comprises at least one member projecting from the body and engageable with the femur.

5. The hip stem centralizer of claim 4 wherein the member engages the outer surface of the femur to position the resilient opening relative to the outer surface.

6. The hip stem centralizer of claim 4 wherein the member is engageable with holes formed in the femur.

7. The hip stem centralizer of claim 4 wherein the member is engageable with the intramedullary canal to locate the resilient opening relative to the intramedullary canal.

8. The hip stem centralizer of claim 7 wherein the member comprises a plurality of tabs extending distally from the bottom of the body in predetermined radial relationship to the resilient opening.

9. The hip stem centralizer of claim 1 wherein the body comprises a relatively more rigid outer ring and a relatively less rigid inner ring mounted inside of the outer ring, the inner ring defining the resilient opening.

10. The hip stem centralizer of claim 9 wherein the outer ring comprises a relatively rigid plastic and the inner ring comprises a relatively less rigid resilient polymer foam.

11. The hip stem centralizer of claim 9 wherein the inner and outer rings are bonded together.

12. The hip stem centralizer of claim 9 wherein the outer ring comprises an outer profile shaped to match the shape of the proximal portion of the femur, the profile being alignable with the proximal femur to position the resilient opening relative to the proximal femur.

13. The hip stem centralizer of claim 9 wherein the means for positioning comprises at least one tab extending distally from the bottom of the body in predetermined radial relationship to the resilient opening and the inner ring is compressible on a lateral side to a fully compressed minimum thickness that extends medially inwardly of the lateral tab.

14. The hip stem centralizer of claim 9 wherein the means for positioning comprises at least one tab extending distally from the bottom of the body in predetermined radial relationship to the resilient opening and the outer ring defines an inner perimeter that overlaps the tab radially inwardly.

15. The hip stem centralizer of claim 1 wherein the resilient opening is able to receive the hip stem component in bone cement sealing engagement and the body is engageable with the proximal end of the femur in bone cement sealing engagement.

16. The hip stem centralizer of claim 1 wherein the resilient opening is able to receive the hip stem component in surface wiping engagement.

17. The hip stem centralizer of claim 1 further comprising an inserter engageable with the hip stem component and with the body of the hip stem centralizer to positively locate the hip stem component relative to the hip stem centralizer.

18. The hip stem centralizer of claim 1 further comprising a cement injector and wherein the resilient opening is engageable with the cement injector in canal sealing relationship to permit pressurization of cement through the resilient opening and into the intramedullary canal.

19. A hip stem centralizer for positioning a proximal portion of a femoral hip stem component at a desired radial position within an intramedullary canal of a femur, the femoral hip stem component and intramedullary canal each having a proximal portion nearer the hip joint, a distal portion nearer the knee joint, an anterior portion toward the front of the body, a posterior portion toward the rear of the body, and a lateral portion further from the midline of the body, the femur having an outer surface adjacent the proximal portion, the hip stem centralizer comprising:
a body comprising:
a relatively more rigid outer ring;
a relatively less rigid inner ring having a top and a bottom and being mounted inside of the outer ring, the inner ring defining a resilient opening extending through the inner ring from the top to the bottom, the resilient opening being radially elastically compressible upon receiving the hip stem component; and
at least one member projecting distally from the body in predetermined radial relationship to the resilient opening.

20. A method of positioning a proximal portion of a femoral hip stem component within an intramedullary canal of a femur, the method comprising:
engaging a hip stem centralizer with the opening of the intramedullary canal, the hip stem centralizer defining a resilient opening;
inserting a hip stem component through the resilient opening and into the intramedullary canal, the resilient opening compressing to bias the hip stem component toward the center of the resilient opening; and
disengaging the hip stem centralizer from the hip stem component once the hip stem component has been positioned.
